(19) United States
(12) Patent Application Publication
(10) Pub. No.: US 2006/0135837 A1
(43) Pub. Date: Jun. 22, 2006

(54) Catalyst comprising a metalic support and process for the production of olefins

(76) Inventors: Ian Raymond Little, Naperville, IL (US); Ian Allan Beattie Reid, London (GB); Vaughan Clifford Williams, Middlesex (GB)

Correspondence Address: NIXON & VANDERHYE, PC, 901 NORTH GLEBE ROAD, 11TH FLOOR, ARLINGTON, VA, 22203, US

(21) Appl. No.: 10/557,697

(22) PCT Filed: May 18, 2004
(86) PCT No.: PCT/GB04/02140

§ 371 Date: Nov. 22, 2005

(30) Foreign Application Priority Data
Jun. 5, 2003 (GB) 0312968.1
Jun. 5, 2003 (GB) 0312970.7

(51) Int. Cl. C07C 5/327; B01J 23/40

(52) U.S. Cl. 585/658; 502/325

(57) ABSTRACT

A catalyst capable of supporting combustion beyond the fuel rich limit of flammability comprising a catalytic component and a metallic support wherein the support is a metallic structured packing comprising a multiplicity of open-ended channels and which has been loaded with a non metallic coating, and a process for the production of an olefin, said process comprising passing a mixture of a hydrocarbon and an oxygen-containing gas over said catalyst to produce an olefin.

The present invention relates to a process for the production of olefins from hydrocarbons in which the hydrocarbons are treated to autothermal cracking.

Autothermal cracking is a route to olefins in which the hydrocarbon feed is mixed with oxygen and passed over an autothermal cracking catalyst. The autothermal cracking catalyst is capable of supporting combustion beyond the fuel rich limit of flammability. Combustion is initiated on the catalyst surface and the heat required to raise the reactants to the process temperature and to carry out the endothermic cracking process is generated in situ. Generally the hydrocarbon feed and the oxygen is passed over a supported catalyst to produce the olefin product. Typically, the catalyst comprises at least one platinum group metal, for example, platinum. The autothermal cracking process is described in EP 332289B; EP-529793B; EP-A-0709446 and WO 00/14035.

The catalyst supports are usually non metallic and are typically ceramic materials, usually in the form of foams, monoliths, pellets, beads, spheres, tablets and/or extrudates. However whilst generally being chemically inert non metallic supports can often be unstable to thermal and physical shock which results in support cracking.

The catalyst support may also be metallic. Due to their malleable nature metallic supports do not exhibit support cracking but are often incapable of withstanding excessive front face temperatures that are produced in the autothermal reactor which leads to oxidation and corrosion.

Consequently there is a need to provide an improved support that is both chemically inert and thermally stable.

It has now been found that the autothermal cracking process can be improved by employing a catalyst with a modified metallic support and which has a structure that provides a low pressure drop in the autothermal reactor.

Accordingly, the present invention provides a catalyst capable of supporting combustion beyond the fuel rich limit of flammability comprising a catalytic component and a metallic support wherein the support is a metallic structured packing comprising a multiplicity of open-ended channels and which has been loaded with a non metallic coating.

The present invention also provides a process for the production of an olefin, said process comprising passing a mixture of a hydrocarbon and an oxygen-containing gas over a catalyst as herein described above to produce said olefin.

Preferably, the catalyst component comprises a Group VIIIB metal. Suitable Group VIIIB metals include platinum, palladium, ruthenium, rhodium, osmium and iridium. Preferably, the Group VIIIB metal is selected from rhodium, platinum, palladium or mixtures thereof. Especially preferred are platinum, palladium or mixtures thereof. Typical Group VIIIB metal loadings range from 0.01 to 50 wt %, preferably, from 0.01 to 20 wt %, and more preferably, from 0.01 to 10 wt %, for example 1-5 wt %, such as 3-5 wt %. Suitably, the first catalyst bed comprises platinum or palladium, especially platinum.

Preferably the catalyst component may be a promoted catalyst component such as a promoted Group VIIIB metal catalyst. The promoter may be selected from the elements of Groups IIIA, IVA and VA of the Periodic Table and mixtures thereof. Alternatively, the promoter may be a transition metal; the transition metal being a different metal to the catalyst component, such as the Group VIIIB metal(s) employed as the catalytic component.

The promoter may also be selected from any of the lanthanide metal oxides.

Preferred Group IIIA metals include Al, Ga, In and Tl. Of these, Ga and In are preferred. Preferred Group IVA metals include Ge, Sn and Pb. Of these, Ge and Sn are preferred, especially Sn. The preferred Group VA metal is Sb. The atomic ratio of Group VIIIB metal to the Group IIIA, IVA or VA metal may be 1:0.1-50.0, preferably, 1:0.1-12.0, such as 1:0.3-5.

Suitable transition metal promoters may be selected from any one or more of Groups IB to VIIIB of the Periodic Table. In particular, transition metals selected from Groups IB, IIB, VIIB, VIIB and VIIIB of the Periodic Table are preferred. Examples of such transition metal promoters include V, Ni, Ta, Cr, Mo, W, Mn, Re, Fe, Ru, Os, Co, Rh, Ir, Ni, Pt, Cu, Ag, Au, Zn, Cd and Hg. Preferred transition metal promoters are Mo, Rh, Ru, Ir, Pt, Cu and Zn, especially Cu. The atomic ratio of the Group VIIIB metal to the transition metal promoter may be 1:0.1-50.0, preferably, 1:0.1-12.0.

Specific examples of promoted Group VIIIB metals for use as the promoted catalyst component include Pt/Ga, Pt/In, Pt/Sn, Pt/Ge, Pt/Cu, Pd/Sn, Pd/Ge, Pd/Cu and Rh/Sn. Where the Group VIIIB metal is Rh, Pt or Pd, the Rh, Pt or Pd may comprise between 0.01 and 5.0 wt %, preferably, between 0.01 and 3.0 wt %, and more preferably, between 0.5 and 3.0 wt-% of the total weight of the catalyst. The atomic ratio of Rh, Pt or Pd to the Group IIIA, IVA, VA or transition metal promoter may be 1:0.1-50.0, preferably, 1:0.1-12.0. For example, atomic ratios of Rh, Pt or Pd to Sn may be 1:0.1 to 50, preferably, 1:0.1-12.0, more preferably, 1:0.2-5.0 and most preferably, 1:0.3-5.0. Atomic ratios of Pt or Pd to Ge may be 1:0.1 to 50, preferably, 1:0.1-12.0, and more preferably, 1:0.5-8.0. Atomic ratios of Pt or Pd to Cu may be 1:0.1-3.0, preferably, 1:0.2-2.0, and more preferably, 1:0.5-1.5.

For the avoidance of doubt, the catalyst component and the promoter may be present in any form, for example, as a metal, or in the form of a metal compound, such as an oxide.

The metallic support may be selected from any suitable metal. Suitable metals may include steel (mild and high carbon), stainless steel, Hastaloy, Ni-Chrome, Inconel, Monel, nickel, copper, iron, platinum, noble metals and their alloys, cobalt, FeCrAlY, NiCrAlY, or any alloy containing Y, Cr, Fe, Ni and Al e.g Kanthal, Incoloy MA956, or CoCrAlY. Small amounts of other elements, such as Si, Ti, Nb, Mo, W, Zr, Mg, Cu, may also be present.

Preferably the metal has a melting point of greater than 1200° C. and most preferably the metal is selected from FeCrAlY, NiCrAlY, CoCrAlY, Ni-Chrome and (any grade of) Inconel and Monel.

The metallic support is a metallic structured packing which comprises a multiplicity of open-ended channels. This structure provides a low pressure drop compared to other types of support, such as extrudates and pellets; when used in an autothermal reactor. This is advantageous since high pressure drop in the autothermal reactor can lead to excessive force being applied to the catalyst, which can lead to structural collapse.

The metallic support may be in the form of a foam but is preferably in the form of a channeled monolith.

The structural dimensions of the support type may also vary.

Wherein the support is in the form of a foam, the foams usually have a pore size in the range of 10 pores per inch (ppi) to 100 ppi and preferably between 30 to 45 ppi. These foams typically have a density of from between 60% to 99% of theoretical density of a fully dense material.

Wherein the support material is in the form of a monolith the monolith is usually provided with regular channels. These channels may be of any suitable shape the preferred ones being square, rectangular, triangular, hexagonal and circular. Preferably the monolith is a honeycomb monolith. Typically the channels do not pass directly through the monolith and usually the channels provide a complex passageway through the monolith. Usually the monolith has between 2000 cpi (cells per inch) to 5 cpi and preferably between 1000 cpi to 10 cpi.

The support preferably comprises a series of blocks or layers that tessellate together to leave no gaps. Preferably these blocks or layers are tiled within the reactor in different directions and most preferably in a manner such that tiles of a layer either above or below do not exactly overlap with any neighbouring layer.

The non metallic coating is usually a ceramic material which may be any oxide or combination of oxides that is stable at high temperatures of, for example, between 600° C. and 1200° C. The ceramic material preferably has a low thermal expansion co-efficient, and is resistant to phase separation at high temperatures.

Suitable ceramic materials include alumina, silica-alumina, a combination of alumina and mullite, lithium aluminium silicate, cordierite, silicon carbide, zirconia toughened alumina, partially stabilized zirconia, fully stabilized zirconia, spinel, chromia, titania, aluminium titanate, or any combination of the above.

The non metallic coating may be loaded onto the metallic support by any method known in the art. In particular the non-metallic coating may be loaded onto the support by any one of the following methods; aluminizing, chemical vapour deposition, sputter coating and washcoating.

Wherein the non metallic coating is alumina, aluminizing deposits aluminium metal onto the surface layer of the metallic support. Usually aluminizing comprises heating the metallic support in a cnicible with aluminium powder. The aluminium deposited upon the surface of the metallic support is then oxidized to form alumina.

Wherein chemical vapour deposition is used to provide a non-metallic coating on the metallic support this usually involves the thermal decomposition of a volatile material onto the surface of a heated metallic support.

Wherein sputter coating is used to provide a non-metallic coating on the metallic support the metallic support is spray coated with a fine particulate material which typically contains some sort of binder such that it sticks to the surface of the support. Sputter coating may be performed by arc or laser ablation.

In a preferred embodiment of the invention washcoating is used to provide a non-metallic coating on the metallic support. Washcoating involves providing a slurry of the non metallic coating which is then poured through/over the metallic support. Typically the slurry of the non metallic coating is a ceramic coating and is preferably an alumina colloidal suspension with a carefully defined viscosity and particle size.

Wherein aluminizing is used the thickness of the non metallic coating is usually between 10-200 μm and preferably between 50-100 μm.

Wherein sputter coating is employed the thickness of the non metallic coating is usually between 10 μm-2 mm and preferably between 0.1-1 mm.

Usually the % weight of coating relative to the weight of support is less than 5 wt %, and preferably less than 3 wt %.

Preferably substantially all of the metallic support is coated with the non metallic coating.

The catalyst component employed in the present invention may be loaded onto the coated metal support by any method known in the art. For example, gel methods and wet-impregnation techniques may be employed. Typically, the support is impregnated with one or more solutions comprising the metals, dried and then calcined in air. The support may be impregnated in one or more steps. Preferably, multiple impregnation steps are employed. The support is preferably dried and calcined between each impregnation, and then subjected to a final calcination, preferably, in air. The calcined support may then be reduced, for example, by heat treatment in a hydrogen atmosphere.

Preferably when the catalyst is positioned within the autothermal cracking reactor a non catalytic resistance zone is located upstream of the catalyst. The resistance zone usually comprises a network of capillaries or channels and most preferably the resistance zone comprises a porous material and advantageously the porous material is a non metal e.g. a ceramic material. Suitable ceramic materials include lithium aluminium silicate (LAS), alumina (α-Al₂O₃), yttria-stabilised zirconia, alumina titanate. A preferred porous material is alpha alumina. The porous material may be in the form of spheres, other granular shapes or ceramic foams. Typically the resistance zone has between 10-60 pores per square inch, preferably between 20-50 pores per square inch and most preferably between 30-45 pores per square inch.

The process of the present invention may be used to convert both liquid and gaseous hydrocarbons into olefins. Suitable liquid hydrocarbons include naphtha, gas oils, vacuum gas oils and mixtures thereof. Preferably, however, gaseous hydrocarbons such as ethane, propane, butane and mixtures thereof are employed. Suitably, the hydrocarbon is a paraffin-containing feed comprising hydrocarbons having at least two carbon atoms.

The hydrocarbon feed is mixed with any suitable oxygen-containing gas. Suitably, the oxygen-containing gas is molecular oxygen, air, and/or mixtures thereof. The oxygen-containing gas may be mixed with an inert gas such as nitrogen or argon.

Additional feed components may be included, if so desired. Suitably, hydrogen, carbon monoxide, carbon dioxide or steam may be co-fed into the reactant stream.

Any molar ratio of hydrocarbon to oxygen-containing gas is suitable provided the desired olefin is produced in the process of the present invention. The preferred stoichiometric ratio of hydrocarbon to oxygen-containing gas is 5 to 16, preferably, 5 to 13.5 times, preferably, 6 to 10 times the stoichiometric ratio of hydrocarbon to oxygen-containing gas required for complete combustion of the hydrocarbon to carbon dioxide and water.

The hydrocarbon is passed over the catalyst at a gas hourly space velocity of greater than 10,000 h⁻¹, preferably above 20,000 h⁻¹ and most preferably, greater than 100,000 h⁻. It will be understood, however, that the optimum gas hourly space velocity will depend upon the pressure and nature of the feed composition.

Preferably, hydrogen is co-fed with the hydrocarbon and oxygen-containing gas into the reaction zone. The molar ratio of hydrogen to oxygen-containing gas can vary over any operable range provided that the desired olefin product is produced. Suitably, the molar ratio of hydrogen to oxygen-containing gas is in the range 0.2 to 4, preferably, in the range 1 to 3.

Hydrogen co-feeds are advantageous because, in the presence of the catalyst, the hydrogen combusts preferentially relative to the hydrocarbon, thereby increasing the olefin selectivity of the overall process.

Preferably, the reactant mixture of hydrocarbon and oxygen-containing gas (and optionally hydrogen co-feed) is preheated prior to contact with the catalyst. Generally, the reactant mixture is preheated to temperatures below the autoignition temperature of the reactant mixture.

Advantageously, a heat exchanger may be employed to preheat the reactant mixture prior to contact with the catalyst. The use of a heat exchanger may allow the reactant mixture to be heated to high preheat temperatures such as temperatures at or above the autoignition temperature of the reactant mixture. The use of high pre-heat temperatures is beneficial in that less oxygen reactant is required which leads to economic savings. Additionally, the use of high preheat temperatures can result in improved selectivity to olefin product. It has also been found that the use of high preheat temperatures enhances the stability of the reaction within the catalyst thereby leading to higher sustainable superficial feed velocities, and also reduces the thermal gradient experienced across the catalyst.

The process of the present invention may suitably be carried out at a catalyst exit temperature in the range 600° C. to 1200° C., preferably, in the range 850° C. to 1050° C. and, most preferably, in the range 900° C. to 1000° C.

The process of the present invention is usually operated at a pressure of greater than 0.5 barg. Preferably the autothermal cracking process is operated at a pressure of between 0.5-40 barg and advantageously between 10-30 barg e.g. 15-25 barg.

The reaction products are preferably quenched as they emerge from the reaction chamber to avoid further reactions taking place. Usually the product stream is cooled to between 750-600° C. within less than 100 milliseconds of formation, preferably within 50 milliseconds of formation and most preferably within 20 milliseconds of formation e.g. within 10 milliseconds of formation.

Wherein the autothermal cracking process is operated at a pressure of 5-20 barg usually the products are quenched and the temperature cooled to between 750-600° C. within 20 milliseconds of formation. Advantageously wherein the autothermal cracking process is operated at a pressure of greater than 20 barg the products are quenched and the temperature cooled to between 750-600° C. within 10 milliseconds of formation.

The invention will now be illustrated by the following examples.

### EXAMPLES

Preparation of Catalysts

Comparative Catalyst 1

FeCrAlY foam blocks, comprising (by weight) approximately 73% iron, 20% chromium, 5% aluminium and 2% yttrium (III) oxide, in the shape of cylinders having dimensions of 15 mm diameter by 25 mm depth, and pore size of 30 pores per inch (ppi) were purchased from Porvair Advanced Materials.

The foams were repeatedly impregnated by immersion in a solution of tetraamineplatinum (II) chloride and copper (II) nitrate hekahydrate, said solution containing sufficient of each respective salt to achieve a nominal Pt loading of 3 wt % and a nominal Cu loading of 1 wt % if all the metal in the respective salts were incorporated into the final catalyst formulation:

Between impregnations excess solution was removed from the foams, which were then dried in air at ca. 120° C. for approximately 20 minutes. After all the metal salts had been incorporated the foams were calcined in air at 600° C. for approximately 6 hours, cooled to room temperature, and then reduced under a flow of 50 vol % hydrogen/50 vol % nitrogen at 750° C. and at a flow rate of 2 nl/min for 1 hour.

Catalyst A

As for Catalyst 1, FeCrAlY foam blocks comprising (by weight) approximately 73% iron, 20% chromium, 5% aluminium and 2% yttrium (III) oxide, in the shape of cylinders having dimensions of 15 mm diameter by 25 mm depth, and having a pore size of 30 pores per inch (ppi) were purchased from Porvair Advanced Materials.

The foams were washcoated with a gamma alumina washcoat and calcined before being loaded with Pt and Cu at a nominal Pt loading of 3 wt % and a nominal Cu loading of 1 wt % (assuming all the metal were incorporated into the final catalyst formulation).

The foams were subsequently calcined in air at 600° C. for approximately 6 hours, cooled to room temperature, and then reduced under a flow of 50 vol % hydrogen/50 vol % nitrogen at 750° C. and at a flow rate of 2 nl/min for one hour.

Catalyst Testing

Catalyst testing was performed at atmospheric pressure (0 barg) in an autothermal reactor comprising a steel reactor in an electrically heated furnace.

The catalyst blocks were positioned in the reactor between two LAS heat shields. Two blocks of catalyst were loaded in sequential fashion into the reactor for each test, to give a total catalyst bed of 50 mm depth, and the reactor heated to 850° C. Ethane (6.09 nl/min), hydrogen (5.48 nl/min), nitrogen (1.61 nl/min) and oxygen (2.74 nl·min) were supplied from cylinders via mass flow controllers into two manifolds, one for oxygen, the second for the other gases. The two gas streams were pre-heated to around 100° C. and then mixed immediately before the catalyst. The product gases were sampled and analysed by gas chromatography. The results are shown in Table 1 and Table 2.

TABLE 1
Ethylene yield (g/100 g hydrocarbon) with time on
stream for Comparative Catalyst 1 and Catalyst A.

Ethylene yield (g/100 g)

| Time on stream hours | Comparative Catalyst 1 | Catalyst A |
|---|---|---|
| 1.75 | — | 56.6 |
| 2.5 | 51.5 | — |
| 4.75 | 51.35 | — |
| 5.0 | — | 56.7 |
| 9.25 | 52.3 | — |
| 21.75 | — | 56.4 |
| 25.2 | 54.5 | — |
| 26.0 | — | 56.3 |
| 32.0 | 54.3 | — |
| 44.5 | — | 56.6 |
| 48.0 | — | 56.7 |
| 48.5 | 55.0 | — |
| 51.7 | — | 56.7 |
| 52.7 | 55.44 | — |
| 58.3 | 53.4 | — |
| 68.5 | — | 56.6 |
| 72.2 | 55.2 | — |
| 74.4 | — | 56.1 |

TABLE 2
Product distribution at 48 hours on stream
for Comparative Catalyst 1 and Catalyst A.

| | Comparative Catalyst 1 | Catalyst A |
|---|---|---|
| Ethane Conversion (%) | 78.8 | 81.0 |

Yield (g/100 g hydrocarbon):

| | | |
|---|---|---|
| Hydrogen | 7.60 | 7.10 |
| Water | 40.2 | 43.2 |
| Methane | 5.27 | 6.71 |
| CO | 14.2 | 14.8 |
| CO2 | 5.68 | 1.58 |
| Ethylene | 55.0 | 56.7 |
| Ethane | 21.2 | 19.0 |
| Acetylene | 0.58 | 0.94 |

Table 1 shows that, relative to a non-washcoated metallic foam, Catalyst A results in an increased ethylene yield. Table 2 shows that, relative to a non-washcoated metallic foam, and in addition to an increased ethylene yield, Catalyst A also results in a significantly reduced carbon dioxide yield.

1-14. (canceled)

15. A process for the production of an olefin, said process comprising passing a mixture of a hydrocarbon and an oxygen-containing gas over a catalyst capable of supporting combustion beyond the fuel rich limit of flammability, said catalyst comprising a catalytic component and a metallic support wherein the support is a metallic structured packing comprising a multiplicity of open-ended channels and which has been loaded with a non metallic coating.

16. A process as claimed in claim 15, wherein the catalyst component comprises a Group VIIIB metal.

17. A process as claimed in claim 15, wherein the metallic support is selected from FeCrAlY, NiCrAlY, CoCrAlY, Ni-Chrome, Inconel and Monel.

18. A process as claimed in claim 15, wherein the metallic support is in the form of a foam having a pore size in the range of 10 pores per inch (ppi) to 1 Ooppi.

19. A process as claimed in claim 15, wherein the metallic support is in the form of a monolith having between 2000 cpi (cells per inch) to 5 cpi.

20. A process as claimed in claim 15, wherein the metallic support comprises a series of blocks or layers that tessellate together to leave no gaps.

21. A process as claimed in claim 15, wherein the non metallic coating is a ceramic material selected from alumina, silica-alumina, a combination of alumina and mullite, lithium aluminium silicate, cordierite, silicon carbide, zirconia toughened alumina, partially stabilized zirconia, fully stabilized zirconia, spinel, chromia, titania, aluminium titanate, or any combination of the above.

22. A process as claimed in claim 15, wherein hydrogen is co-fed with the hydrocarbon and oxygen-containing gas to the reaction zone.

23. A process as claimed in claim 15, wherein a non catalytic resistance zone is located upstream of the catalyst.

24. A process as claimed in claim 15, wherein the ratio of hydrocarbon to oxygen-containing gas is 5 to 16, times the stoichiometric ratio of hydrocarbon to oxygen-containing gas required for complete combustion of the hydrocarbon to carbon dioxide and water.

25. A process as claimed in claim 15, wherein the process is operated at a pressure of between 10-30 barg.
